(19) United States
(12) Patent Application Publication
(10) Pub. No.: US 2025/0221896 A1
(43) Pub. Date: Jul. 10, 2025

(54) COSMETIC COMPOSITIONS INCLUDING CALCIUM CARBONATE PARTICLES AND GRAPHENE

(71) Applicant: Nant Holdings IP, LLC, Culver City, CA (US)

(72) Inventor: Patrick SOON-SHIONG, Los Angeles, CA (US)

(21) Appl. No.: 19/004,856

(22) Filed: Dec. 30, 2024

Related U.S. Application Data
(60) Provisional application No. 63/618,020, filed on Jan. 5, 2024.

(51) Int. Cl. A61K 8/19; A61K 8/06; A61Q 1/10

(52) U.S. Cl. CPC A61K 8/19; A61K 8/06; A61Q 1/10; A61K 2800/412; A61K 2800/43; A61K 2800/87

(57) ABSTRACT

Cosmetic compositions, such as a mascara and/or an eyeliner, are provided herein, which include graphene and calcium carbonite particles, such as aragonite particles. Also provided are methods of using the cosmetic compositions and devices including the cosmetic compositions.

### CROSS-REFERENCE TO RELATED APPLICATIONS

This patent application claims the benefit of U.S. Provisional Patent Application Ser. No. 63/618,020, filed on 5 Jan. 2024, the entire contents of which are incorporated herein by reference.

### FIELD

This disclosure generally relates to cosmetic compositions including calcium carbonate particles and graphene and methods and devices for applying the cosmetic compositions. The cosmetic compositions, such as a mascara composition or an eyeliner composition, may include calcium carbonate particles, such as aragonite particles, and graphene.

### BACKGROUND

This section provides background information related to the present disclosure which is not necessarily prior art.

Cosmetic compositions for application to the eye area, such as a mascara, an eyebrow pencil, and an eyeliner, may be used to accentuate various parts of the eye. For example, a mascara may be used to thicken, lengthen, color, curl, and/or provide volume to eyelashes or eyebrows and an eyebrow pencil or eyeliner may be used to define, thicken, and/or color eyebrows or eyelids. Mascaras are generally based on emulsifying systems associated with oils, waxes or lipophilic substances and may also include polymeric and/or fibrous components for defining, thickening, and lengthening of eyelashes and eyebrows as well as dyes or pigments to provide color. Exemplary mascara formulations include water-resistant, waterproof, and volumizing formulations as oil in water, water in oil, or anhydrous formulations. A common type of mascara formulation is based on an oil in water emulsion, such as a lamellar gel network, which is stabilized using soluble polymers.

U.S. Pat. No. 11,273,107 reports an eye make-up cosmetic composition including graphene, which is free of synthetic polymers.

Some mascara compositions can be prone to smearing and smudging after application and the solid components dispersed in the composition, such as waxes, may also be difficult to apply, as clumping and globbing may occur due to lack of film smoothness of the solid components. With regard to eyeliners and eyebrow pencils, compositions are needed that can provide high definition upon application and ease of distribution on the area applied, for example, the upper and/or lower eyelid. Further, eyebrow pencils and eyeliners that do not release color over time or smudge in the presence of moisture or water are needed, especially as eyeliners can be applied to areas of the eye subject to lacrimation. Thus, improved and alternative cosmetic formulations for application to the eye area are needed.

### SUMMARY

This section provides a general summary of the disclosure and is not a comprehensive disclosure of its full scope or all of its features.

In certain aspects, the present disclosure provides a cosmetic composition, such as a mascara composition, eyeliner composition and/or eyebrow pencil composition. The cosmetic composition includes calcium carbonate particles, such as aragonite particles, and graphene. The cosmetic composition may further include at least one of a dye and an emulsifier.

In yet other aspects, the present disclosure provides a further cosmetic composition, such as a mascara, eyeliner and/or eyebrow pencil, which includes calcium carbonate particles, such as aragonite particles, and graphene and which does not include a bituminous conglomerate.

In yet other aspects, the present disclosure provides a method for thickening the appearance of eyelashes and/or eyebrows and/or increasing the volume of the eyelashes and/or the eyebrows. The method includes applying the cosmetic composition as described herein to the eyelashes and/or the eyebrows.

In yet other aspects, the present disclosure provides a device for applying mascara or eyeliner. The device includes a container, the cosmetic composition as described herein within the container, and an applicator for applying the mascara or eyeliner to eyelashes, eyebrows and/or the eyelid. The applicator is detachable from the container.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 illustrates one non-limiting embodiment of a device for applying mascara or eyeliner in accordance with the present disclosure.

### DETAILED DESCRIPTION

Example embodiments are provided so that this disclosure will be thorough and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

### A. Definitions

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this disclosure, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time.

In addition, disclosure of ranges includes disclosure of all values and further divided ranges within the entire range, including endpoints and sub-ranges given for the ranges.

### B. Cosmetic Compositions

Cosmetic compositions for application to an eye area, which include calcium carbonate (CaCO₃) and graphene are provided herein. The calcium carbonate may be naturally occurring calcium carbonate or synthesized calcium carbonate. Calcium carbonate may be naturally found as a mineral calcite and/or aragonite in materials, such as chalk, limestone, marble, eggshells, gastropod shells, shellfish skeletons and pearls. Additionally, the calcium carbonate may be present in the form of particles.

In any embodiment, the calcium carbonate particles can comprise aragonite particles. Aragonite, a natural orthorhombic crystalline form of calcium carbonate, occurs most commonly in beds of gypsum and of iron ore. Aragonite differs from calcite in that has an orthorhombic crystalline structure, a greater specific gravity (2.93 to 2.95 g/cm³ as compared to 2.71 g/cm³ for calcite), and less distinct cleavage than calcite.

One form of aragonite, known as “oolitic aragonite,” occurs on the ocean floor throughout the world. Oolitic aragonite occurs in discrete grains which are essentially spherical in form. The material as a marine deposit is unconsolidated and varies in grain size with varying amounts of shell fragments intermixed therein. Oolitic aragonite can be found in the Caribbean, for example, on and around the Bahama islands. An exemplary composition of oolitic aragonite is provided in Table 1 below.

TABLE 1

| Composition | Percent by Weight |
|---|---|
| CaCO₃ | 97.00 |
| SiO₂ | 0.04 |
| Fe₂O₃ | 0.02 |
| Al₂O₃ | 0.02 |
| MgO | 0.23 |
| Mn | 0.0005 to 0.005 |
| Sr | 0.1 to 1.0 |
| S (organic) | 0.13 |
| S (inorganic) | 0.01 |
| Chloride (as NaCl) | 0.25 |
| Other organic matter | 0.41 |

Oolitic aragonite may be recovered from the ocean floor, for example, by dredging or otherwise removing the oolitic aragonite from the ocean floor. Dredging of the ocean floor can take place near, adjacent to, or at a beach in the Bahama Islands. The recovered oolitic aragonite can include various sizes of material. In any embodiment, the recovered oolitic aragonite can have a particular size diameter from about 50 μm to about 2 mm. In some embodiments, oolitic aragonite can first be screened to remove any oversized material, such as shell fragments having a particle size diameter greater than or equal to about 275 μm. Alternatively, oolitic aragonite can be used directly from the ocean without any screening, grinding, or both. In other words, the oolitic aragonite can be unprocessed when incorporated into a cosmetic composition. Thus, the term “unprocessed” as used herein, means no further treatment to the aragonite after its recovery from the ocean floor other than the mere handling and transport of it to a stockpile and then incorporating it into a cosmetic composition. In any embodiment, the aragonite (e.g., oolitic aragonite) is substantially dry, for example, containing less than or equal to about 10%, less than or equal to about 5%, less than or equal to about 2.5%, less than or equal to about 1%, less than or equal to about 0.5%, or less than or equal to about 0.2% moisture content, prior to incorporation into a cosmetic composition.

In any embodiment, at least 30%, at least 50%, at least 70%, or at least 90% of the calcium carbonate particles, such as aragonite particles (e.g., oolitic aragonite), may have an average particle size in diameter of greater than or equal to about 1 nm, greater than or equal to about 50 nm, greater than or equal to about 100 nm, greater than or equal to about 500 nm, greater than or equal to about 1 μm, greater than or equal to about 3 μm, greater than or equal to about 5 μm, greater than or equal to about 10 μm, greater than or equal to about 50 μm, greater than or equal to about 100 μm, less than or equal to about 2 mm, less than or equal to about 1 mm, less than or equal to about 750 μm, less than or equal to about 500 μm, or less than or equal to about 250 μm, or greater than or equal to about 1 nm to less than or equal to about 2 mm, greater than or equal to about 50 nm to less than or equal to about 2 mm, greater than or equal to about 100 nm to less than or equal to about 1 mm, greater than or equal to about 1 μm to less than or equal to about 500 μm, greater than or equal to about 10 μm to less than or equal to about 100 μm, greater than or equal to 1 nm to less than or equal to 100 μm, greater than or equal to 10 nm to less than or equal to 1 μm, or greater than or equal to 100 nm to less than or equal to 500 nm. As used herein, “diameter” encompasses the longest transverse distance through the particle that passes through the particle's center of mass.

In any embodiment, at least a portion of or substantially all of the calcium carbonate particles, such as aragonite particles (e.g., oolitic aragonite), may be ground prior to incorporation into a cosmetic composition, for example, in a ball mill. Suitable milling techniques and milled aragonite particles are described in U.S. Pat. No. 11,383,988, issued on 12 Jul. 2022, the entire contents of which are herein incorporated by reference. The calcium carbonate particles, such as aragonite particles (e.g., oolitic aragonite), may be ground or milled to particles having an average particle size in diameter of greater than or equal to about 1 μm, greater than or equal to about 3 μm, greater than or equal to about 5 μm, greater than or equal to about 8 μm, greater than or equal to about 10 μm, greater than or equal to about 13 μm, greater than or equal to about 15 μm, greater than or equal to about 18 μm, or about 20 μm; or from about 1 μm to about 20 μm, about 1 μm to about 15 μm, about 3 μm to about 10 μm, about 3 μm to about 8 μm, or about 3 μm to about 5 μm. Additionally or alternatively, the calcium carbonate particles, such as aragonite particles (e.g., oolitic aragonite), may have a particle size distribution (PSD) of greater than or equal to about 2 μm to less than or equal to about 3.5 μm. It is also contemplated herein that calcium carbonate particles, such as aragonite particles (e.g., oolitic aragonite), do not undergo any further processing such as milling and may be considered unprocessed prior to incorporation into the cosmetic composition.

The calcium carbonate particles, such as aragonite particles (e.g., oolitic aragonite), may be present in the cosmetic composition in an amount, based on total weight of the composition, of greater than or equal to about 0.1%, greater than or equal to about 0.5%, greater than or equal to about 1%, greater than or equal to about 5%, greater than or equal to about 10%, greater than or equal to about 15%, less than or equal to about 75%, less than or equal to about 50%, less than or equal to about 30%, or less than or equal to about 25%, or from greater than or equal to about 0.1% to less than or equal to about 75%, greater than or equal to about 0.5% to less than or equal to about 50%, greater than or equal to about 1% to less than or equal to about 30%, or greater than or equal to about 5% to less than or equal to about 25%.

The graphene present in the cosmetic composition may be recycled graphene, virgin graphene, or a mixture of recycled graphene and virgin graphene. The recycled and/or virgin graphene may be in the form of graphene particles, graphene nanoplatelets, or a combination thereof. Graphene nanoplatelets are formed from a physical transformation of natural graphite. Graphene nanoplatelets have a high aspect ratio, have high lateral dimension (in the micron (μm) range), and a low thickness (in the nanometer (nm) range). For example, suitable commercially available graphene includes G+® Graphene Plus materials available from Directa Plus S.p.A, such as GRAFYSORBER®, LIQUID®, MICRO G+R, PASTE GIR, AND PURE GIR. In some embodiments, the graphene may be used in the form of a paste. The graphene may be present in the cosmetic composition in an amount, based on total weight of the composition, of greater than or equal to about 1%, greater than or equal to about 5%, greater than or equal to about 7.5%, greater than or equal to about 10%, less than or equal to about 40%, less than or equal to about 30%, less than or equal to about 20%, or less than or equal to about 15%, or from greater than or equal to about 1% to less than or equal to about 40%, greater than or equal to about 1% to less than or equal to about 30%, greater than or equal to about 5% to less than or equal to about 15%, or greater than or equal to about 7.5% to less than or equal to about 10%.

In various aspects, the graphene is hydrophobic and may range from weakly hydrophobic to strongly hydrophobic. A water contact angle with a surface of greater than or equal to about 60° to less than about 90° is considered weakly hydrophobic. A water contact angle with a surface of greater than or equal to about 90° to less than about 150° is considered hydrophobic or normally hydrophobic. A water contact angle with a surface of greater than or equal to about 150° to less than or equal to about 180° is considered strongly hydrophobic. In any embodiment, graphene may have a water contact angle with a surface of greater than or equal to about 60°, greater than or equal to about 75°, greater than or equal to about 90°, greater than or equal to about 110°, greater than or equal to about 125°, less than or equal to about 180°, less than or equal to about 160°, less than or equal to about 150°, or less than or equal to about 140°, or from greater than or equal to about 60° to less than or equal to about 180°, greater than or equal to about 90° to less than or equal to about 180°, greater than or equal to about 110° to less than or equal to about 180°, or greater than or equal to about 125° to less than or equal to about 180°. This hydrophobic nature of graphene can advantageously result in improved endurance of the cosmetic composition on the application area (e.g., skin, eyelashes, eyebrows) and avoid smudging of the composition during wear.

Additionally or alternatively, at least some of the calcium carbonate particles, such as aragonite particles (e.g., oolitic aragonite), may be coupled to the graphene. For example, at least some of the calcium carbonate particles are at least partially encapsulated by the graphene, or at least some of the calcium carbonate particles are present within a structure of the graphene, or at least some of the graphene is present between one or more layers of calcium carbonate particles, or any combination thereof. The calcium carbonate particles can form a thin sheet or layer and a plurality of graphene can be placed in between the sheets or layers of calcium carbonate particles (e.g., sandwich structure). Alternatively, calcium carbonate particles can be encapsulated or captured inside the hollow spaces created by the web-like structure of graphene. The calcium carbon particles may be combined or mixed with graphene in any suitable ratio, for example, a 1:1 ratio, at 2:1 or 1:2 ratio, a 5:1 or 1:5 ratio, a 10:1 or 1:10 ratio, a 20:1 or 1:20 ratio, a 30:1 or a 1:30 ratio, etc.

In any embodiment, a cosmetic composition may include one or more additional components, such at least one of an emulsifier and a dye. Suitable examples of an emulsifier include, but are not limited to, palmitic acid, stearic acid, isostearic acid, oleic acid, potassium cetyl phosphate, 2-(octadecyloxy) ethanol, polyethylene glycol octadecyl ether, polyethylene glycol 1100 mono(hexadecyl/octadecyl) ether, glyceryl stearate, glyceryl behenate, cetyl alcohol, myristyl alcohol, stearyl alcohol, or a combination thereof. An emulsifier may be present in the cosmetic composition in an amount, based on total weight of the composition, of greater than or equal to about 0% to less than or equal to about 20%, greater than or equal to about 1% to less than or equal to about 10%, or greater than or equal to about 4% to less than or equal to about 8%.

Suitable examples of a dye include, but are not limited to, a mineral pigment, an organic pigment, or a combination thereof. Pigments can be white or colored. Pigments can also be pearly. Exemplary mineral pigments include titanium dioxide, coated titanium dioxide, iron oxides, chrome oxides, manganese violet, ultramarine, etc. Exemplary organic pigments include carbon black. An example of black dye is a mixture C.I. 77266, Ceteareth-25, sodium carbonate, sorbitol, citric acid. Examples of white pearly pigments are mica coated with titanium dioxide or bismuth oxychloride. An example of colored pigment is mica with titanium dioxide, iron oxides, etc. A pigment may be present in the cosmetic composition in an amount, based on total weight of the composition, of greater than or equal to about 0% to less than or equal to about 20%, greater than or equal to about 1% to less than or equal to about 20%, or greater than or equal to about 5% to less than or equal to about 10%.

Additionally or alternatively, the cosmetic composition may include one or more additives, such as, but not limited to, a solvent, a wax, a polymer, a humectant, a preservative, a dispersing agent, an emulsion stabilizer, a lash lengthening fiber, a volumizing powder, an oil, or a combination thereof. Exemplary solvents include, but are not limited to, an alcohol (e.g., ethanol), isododecane, cyclopentasiloxane, and combinations thereof. Exemplary waxes include, but are not limited to, carnauba wax, synthetic wax, beeswax, polyethylene, spermaceti, lanolin, shellac, candelilla wax, sugar cane wax, rice wax, olive wax, and combinations thereof. Exemplary polymers include, but are not limited to, ammonium acrylates copolymer, acrylates copolymer, acrylates/styrene/ammonium methacrylate copolymer, polyvinylpyrrolidone (PVP), vinylpyrrolidone/eicosane copolymer, ethylenediamine/stearyl dimer dilinoleate copolymer, polyethylene, alkyl acrylates, alkyl methacrylates, polyacrylates, polymethacrylates, polyolefins, polyvinyls, polyurethanes, polyamides, polyimides, polyethers, polyesters, fluoropolymers, polyacetates, polycarbonates, epoxides, aldehyde resins, polysiloxanes, polyquaterniums, and combinations thereof. Exemplary humectants include, but are not limited to, propanediol, butylene glycol, and combinations thereof. Exemplary preservatives include, but are not limited to, phenoxyethanol, caprylyl glycol, hexanediol, pentylene glycol, ethylhexylglycerin, disodium/trisodium ethylenediaminetetraacetic acid (EDTA), sodium dehydroacetic acid, benzoic acid, sorbic acid, benzyl alcohol, dehydroacetic acid, anisic acid, tocopherol and derivatives thereof, and combinations thereof. Exemplary dispersing agents include, but are not limited to, lecithin, sodium polymethacrylate, sodium polyacrylate, and combinations thereof. Exemplary emulsion stabilizers include, but are not limited to, xanthan gum, hydroxyethylcellulose, magnesium aluminum silicate, and combinations thereof. Exemplary lash lengthening fibers and volumizing powders include, but are not limited to, nylon 6, rayon, vegetal fibers, and combinations thereof. Exemplary oils include, but are not limited to, triglycerides of caprylic/capric acid, tricaprylin, isoamyl laurate, vegetal oils, such as castor oil, sesame, linen, coconut, corn, cotton seeds, olive, palm, illipe, rape, soy, sunflower, walnut, avocado, camellia, macadamia walnut, grapes seeds, peanut, and jojoba oils, and combinations thereof. Further additives contemplated herein include one or more of glyceryl behenate, glyceryl stearate, perfumes, emollients, sequestrants, neutralizing agents, sun filters, sprays, fillers, rheology modifiers, pH correctors, moisteners (e.g. glycerin), moisturizers, vitamins, hyaluronic acid, etc. Additionally, water may be used as a carrier in the cosmetic composition, for example, in an emulsified phase, such as oil-in-water emulsion.

Each of the above-described additives independently may be present in the cosmetic composition in an amount, based on total weight of the cosmetic composition, of greater than or equal to about 0% to less than or equal to about 20%, greater than or equal to about 1% to less than or equal to about 20%, or greater than or equal to about 5% to less than or equal to about 10%. For example, the following additive amounts, based on total weight of the cosmetic composition, may be present: greater than or equal to about 1% to less than or equal to about 10% solvent(s); greater than or equal to about 1% to less than or equal to about 10% wax(es); greater than or equal to about 1% to less than or equal to about 5% polymer(s); greater than or equal to about 1% to less than or equal to about 5% humectant(s); greater than or equal to about 0.5% to less than or equal to about 5% preservative(s), greater than or equal to about 0% to less than or equal to about 2.5% dispersing agent(s); greater than or equal to about 0.1% to less than or equal to about 2.5% emulsion stabilizer(s); greater than or equal to about 0% to less than or equal to about 5% lash lengthening fiber(s)/volumizing powder(s); and greater than or equal to about 2% to less than or equal to about 20% oil(s).

It is also contemplated herein that the cosmetic composition does not include components unsuitable for or incompatible with cosmetic applications. For example, the cosmetic composition does not include a bituminous conglomerate. As used herein, “bituminous conglomerate” refers to a composition comprising bitumen or modified bitumen, also known as “asphalt.”

The cosmetic compositions provided herein may be used as a mascara, an eyeliner, and/or eyebrow pencil. Thus, the cosmetic compositions may be applied to eyelashes, eyebrows, eyelids and/or skin around eyelids. The use of the cosmetic composition as a mascara for eyelashes can achieve increased volume and length of the eyelashes with good separation between the eyelashes and an optimal curling of the eyelashes, which can be free from smudging and clumps. If applied to the eyebrows, the cosmetic composition can provide a volumizing and thickening effect capable of dying the eyebrows. When used as an eyeliner and/or eyebrow pencil, the cosmetic composition can be easily applied and distributed while maintaining high definition as well as ensuring long-lasting wear.

### C. Methods and Devices Using the Cosmetic Compositions

Methods of thickening the appearance of eyelashes and/or eyebrows and/or increasing the volume of the eyelashes and/or the eyebrows are provided. The methods may include applying the cosmetic composition as described herein to the eyelashes and/or the eyebrows. Any suitable cosmetic device may be used to apply the cosmetic composition to the eyelashes and/or the eyebrows. For example, an applicator, such as a wand, including a bristled end loaded with the cosmetic composition may be applied to the eyelashes and/or the eyebrows via one or more applications as needed.

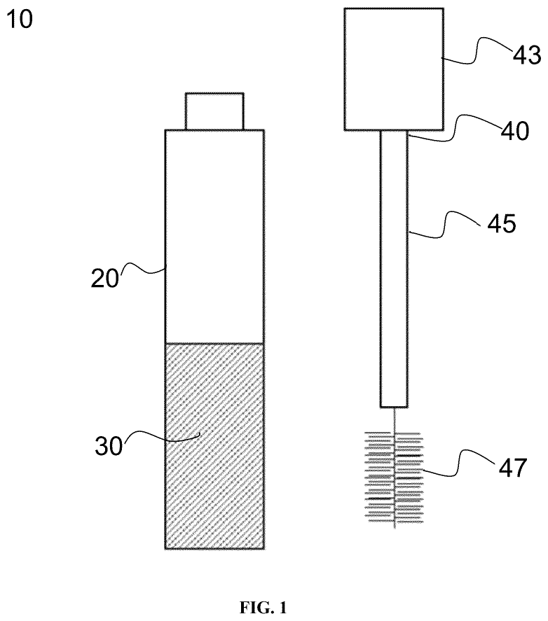

A device for applying mascara or eyeliner is also provided. An exemplary illustration of the device is depicted in FIG. 1. As shown in FIG. 1, the device 10 includes a container 20, the cosmetic composition 30 as described herein present within the container 20, and an applicator 40 for applying the mascara or eyeliner to eyelashes, eyebrows, and/or the eyelid. The applicator 40 is detachable from the container 20. The applicator 40 may include a cap 43, a shaft 45, and a bristled end 47 at the end of the shaft 45 for loading the cosmetic composition 30 thereon when the applicator 40 is inserted into the container 20. Although applicator 40 is shown with the bristled end 47 in line with the shaft 45, it is contemplated herein that the bristled end 47 may be positioned at an angle to the shaft 45. Although not shown, the bristled end 47 may be replaced with a brush for applying the cosmetic composition.

Although the present disclosure has been described in conjunction with the specific drawings and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A cosmetic composition comprising:
calcium carbonate particles;
graphene; and
at least one of a dye and an emulsifier.

2. The cosmetic composition of claim 1, wherein the calcium carbonate particles comprise aragonite particles.

3. The cosmetic composition of claim 2, wherein the aragonite particles have an average particle size of greater than or equal to about 100 nm to less than or equal to about 1 mm in diameter and/or the aragonite particles have a particle size distribution (PSD) of greater than or equal to about 2 μm to less than or equal to about 3.5 μm.

4. The cosmetic composition of claim 2, wherein the aragonite particles comprise oolitic aragonite particles.

5. The cosmetic composition of claim 4, wherein the oolitic aragonite particles comprise milled oolitic aragonite particles.

6. The cosmetic composition of claim 1, wherein the graphene satisfies one or more of the following:
(i) the graphene is present in an amount of greater than or equal to about 5% to less than or equal to about 15%, by weight, based on total weight of the composition;
(ii) the graphene is hydrophobic having a water contact angle (WCA) of greater than or equal to about 90°; and
(iii) the graphene is in the form of graphene nanoplatelets or particles.

7. The cosmetic composition of claim 1, wherein at least some of the calcium carbonate particles are coupled to the graphene, or at least some of the calcium carbonate particles are at least partially encapsulated by the graphene, or at least some of the calcium carbonate particles are present within a structure of the graphene, or at least some of the graphene is present between one or more layers of calcium carbonate particles, or a combination thereof.

8. The cosmetic composition of claim 1, wherein the emulsifier comprises palmitic acid, stearic acid, isostearic acid, oleic acid, potassium cetyl phosphate, 2-(octadecyloxy) ethanol, polyethylene glycol octadecyl ether, polyethylene glycol 1100 mono(hexadecyl/octadecyl) ether, glyceryl stearate, glyceryl behenate, cetyl alcohol, myristyl alcohol, stearyl alcohol, or a combination thereof, and wherein the dye comprises a mineral pigment, an organic pigment, or a combination thereof.

9. The cosmetic composition of claim 1, further comprising one or more additives comprising a solvent, a wax, a polymer, a humectant, a preservative, a dispersing agent, an emulsion stabilizer, a lash lengthening fiber, a volumizing powder, an oil, or a combination thereof.

10. The cosmetic composition of claim 1, wherein the cosmetic composition is a mascara, an eyeliner, or an eyebrow pencil.

11. The cosmetic composition of claim 1, wherein the cosmetic composition does not include a bituminous conglomerate.

12. A cosmetic composition comprising:
calcium carbonate particles; and
graphene,
wherein the cosmetic composition does not include a bituminous conglomerate.

13. The cosmetic composition of claim 12, wherein the calcium carbonate particles comprise aragonite particles.

14. The cosmetic composition of claim 13, wherein the aragonite particles have an average particle size of greater than or equal to about 100 nm to less than or equal to about 1 mm in diameter and/or the aragonite particles have a particle size distribution (PSD) of greater than or equal to about 2 μm to less than or equal to about 3.5 μm.

15. The cosmetic composition of claim 13, wherein the aragonite particles comprise oolitic aragonite particles.

16. The cosmetic composition of claim 15, wherein the oolitic aragonite particles comprise milled oolitic aragonite particles.

17. The cosmetic composition of claim 12, wherein the graphene satisfies one or more of the following:
(iv) the graphene is present in an amount of greater than or equal to about 5% to less than or equal to about 15%, by weight, based on total weight of the composition;
(v) the graphene is hydrophobic having a water contact angle (WCA) of greater than or equal to about 90°; and
(vi) the graphene is in the form of graphene nanoplatelets or particles.

18. The cosmetic composition of claim 12, wherein at least some of the calcium carbonate particles are coupled to the graphene, or at least some of the calcium carbonate particles are at least partially encapsulated by the graphene, or at least some of the calcium carbonate particles are present within a structure of the graphene, or at least some of the graphene is present between one or more layers of calcium carbonate particles, or a combination thereof.

19. A method of thickening the appearance of eyelashes and/or eyebrows and/or increasing the volume of the eyelashes and/or the eyebrows, the method comprising applying the cosmetic composition of claim 1 to the eyelashes and/or the eyebrows.

20. A device for applying mascara or eyeliner, the device comprising: a container, the cosmetic composition of claim 1 within the container, and an applicator for applying the mascara or eyeliner to eyelashes and/or the eyelid, wherein the applicator is detachable from the container.
